(19) United States
(12) Patent Application Publication
(10) Pub. No.: US 2024/0228390 A1
(43) Pub. Date: Jul. 11, 2024

(54) Chelated Nutrients and Methods of Use Thereof

(71) Applicant: ICL AMERICA DO SUL S.A., São Paulo (BR)

(72) Inventors: Bruno Orui Saito, Paulínia (BR); Daniela Cristina Clemente Vitti, Piracicaba (BR); Michel Castellani da Rocha, Piracicaba (BR); Maria Emilia Moraes Castanho de Almeida, Mogi das Cruzes (BR); Keila Cristina Marques da Silva, Suzano (BR)

(73) Assignee: ICL AMERICA DO SUL S.A., São Paulo, SP (BR)

(21) Appl. No.: 18/569,079

(22) PCT Filed: Jun. 10, 2022
(86) PCT No.: PCT/BR2022/050210

§ 371 Date: Dec. 11, 2023

Related U.S. Application Data
(60) Provisional application No. 63/202,410, filed on Jun. 10, 2021.

(51) Int. Cl. C05D 3/00; C07F 1/00; C07F 3/00; C07F 3/02; C07F 3/06; C07F 13/00; C07F 15/02; C07F 15/04; C07F 15/06

(52) U.S. Cl. CPC C05D 3/00; C07F 1/005; C07F 3/003; C07F 3/02; C07F 3/06; C07F 13/005; C07F 15/025; C07F 15/045; C07F 15/065

(57) ABSTRACT

The present disclosure is directed to chelated nutrients having a structure [M(Ligand 1)(Ligand 2)]2Y, wherein: M is a metal selected from the group consisting of Cu. Mn. Zn. Co. Ni. Mg. Ca and Fe: Ligand 1 is glutamic acid: Ligand 2 is malic acid; and Y is a base cation, wherein the base is selected from the group consisting of NaOH, KOH and NH4OH. And chelated nutrients having a structure [M(Ligand 1)(Ligand 2)]2Y, wherein: M is a metal selected from the group consisting of Cu, Mn, Zn, Co, Ni, Mg, Ca and Fe: Ligand 1 is glutamic acid: Ligand 2 is aspartic acid: and Y is a base cation, wherein the base is selected from the group consisting of NaOH, KOH and NH4OH. The present disclosure is further directed to formulations and methods including the chelated nutrients.

### FIELD OF THE DISCLOSURE

The field of the disclosure relates generally to chelated nutrients and methods and use thereof. More specifically, the disclosure is directed to formulations comprising new nutrients chelated for agronomical applications.

### BACKGROUND OF THE DISCLOSURE

The most common chelating agents (i.e., precursors of chelates) today are EDTA (ethylenediamine tetraacetic acid) and EDDHA (ethylenediaminedi (o-hydroxyphenylacetic) acid). While market demand is high for innovative products that provide a guaranteed source of nutrients for agronomical application, EDTA is most commonly applied for this purpose and already dominates manufacturing technology in several countries. However, there is no record of product biodegradability and bioavailability of conventional chelates within the plant when absorbed.

Accordingly, there is a need for biodegradable and bioavailable chelating agents capable of providing nutrients concentrations in agronomical applications that are also efficient, compatible with other crops products and cost effective, and for instance can be used in addition to or as an alternative to conventional chelates and chelating agents.

### BRIEF DESCRIPTION OF THE DISCLOSURE

In one aspect, the present disclosure is directed to a chelate A, having a structure [M(Ligand 1)(Ligand 2)]2Y, wherein: M is a metal selected from the group consisting of Cu, Mn, Zn, Co, Ni, Mg, Ca and Fe; Ligand 1 is glutamic acid; Ligand 2 is malic acid; and Y is a base cation, wherein the base is preferably selected from the group consisting of NaOH, KOH and NH4OH; and wherein a binder proportion ratio of glutamic acid to malic acid to metal is 1:1:1.

In another aspect, the present disclosure is directed to a chelate B, having a structure [M(Ligand 1)(Ligand 2)]2Y, wherein: M is a metal selected from the group consisting of Cu, Mn, Zn, Co, Ni, Mg, Ca and Fe; Ligand 1 is glutamic acid; Ligand 2 is aspartic acid; and Y is a base cation, wherein the base is preferably selected from the group consisting of NaOH, KOH and NH4OH; and wherein a binder proportion ratio of glutamic acid to aspartic acid to metal is 1:1:1.

In some embodiments of the chelate, Cu is present in an amount between 5 to 7% (liquid form), Mn is present in an amount between 5 to 7% (liquid form), Zn is present in an amount between 5 to 7% (liquid form), Mg is present in an amount between 1.5 to 3.5% (liquid form), Co is present in an amount between 3.5 to 5.5% (liquid form), Ni is present in an amount between 3.5 to 5.5% (liquid form), Ca is present in an amount between 5 to 7% (liquid form), and/or Fe is present in an amount between 5 to 7% (liquid form). In some embodiments, Cu is present in an amount between 13 to 16.5% (solid form), Mn is present in an amount between 13 to 16.5% (solid form), Zn is present in an amount between 13 to 16.5% (solid form), Mg is present in an amount between 6 to 7% (solid form), Co is present in an amount between 13 to 16.5% (solid form), Ni is present in an amount between 13 to 16.5% (solid form), Ca is present in an amount between 13 to 16.5% (solid form), and/or Fe is present in an amount between 13 to 16.5% (solid form).

In another aspect, the present disclosure is directed to a formulation for agronomical application , the formulation comprising: a chelate A having a structure [M(Ligand 1)(Ligand 2)]2Y, wherein: M is a metal selected from the group consisting of Cu, Mn, Zn, Co, Ni, Mg, Ca and Fe; Ligand 1 is glutamic acid; Ligand 2 is malic acid; and Y is a base cation, wherein the base is preferably selected from the group consisting of NaOH, KOH and NH4OH; and water; wherein a binder proportion ratio of glutamic acid to malic acid to metal is 1:1:1.

In another aspect, the present disclosure is directed to a formulation for agronomical application , the formulation comprising: a chelate B having a structure [M(Ligand 1)(Ligand 2)]2Y, wherein: M is a metal selected from the group consisting of Cu, Mn, Zn, Co, Ni, Mg, Ca and Fe; Ligand 1 is glutamic acid; Ligand 2 is aspartic acid; and Y is a base cation, wherein the base is preferably selected from the group consisting of NaOH, KOH and NH4OH; and water; wherein a binder proportion ratio of glutamic acid to aspartic acid to metal is 1:1:1.

In another aspect, the present disclosure is directed to a formulation for agronomical application, the formulation comprising a combination of the chelates A and B.

In some embodiments of the formulation, Cu is present in an amount between 5 to 7% (liquid form), Mn is present in an amount between 5 to 7% (liquid form), Zn is present in an amount between 5 to 7% (liquid form), Mg is present in an amount between 1.5 to 3.5% (liquid form), Co is present in an amount between 3.5 to 5.5% (liquid form), Ni is present in an amount between 3.5 to 5.5% (liquid form), Ca is present in an amount between 5 to 7% (liquid form), and/or Fe is present in an amount between 5 to 7% (liquid form). In some embodiments, Cu is present in an amount between 13 to 16.5% (solid form), Mn is present in an amount between 13 to 16.5% (solid form), Zn is present in an amount between 13 to 16.5% (solid form), Mg is present in an amount between 6 to 7% (solid form), Co is present in an amount between 13 to 16.5% (solid form), Ni is present in an amount between 13 to 16.5% (solid form), Ca is present in an amount between 13 to 16.5% (solid form), and/or Fe is present in an amount between 13 to 16.5% (solid form).

In yet another aspect, the present disclosure is directed to a method of applying an agronomical formulation, the method comprising: preparing a formulation comprising a water soluble chelate A and/or B having a structure [M(Ligand 1)(Ligand 2)]2Y, wherein: M is a metal selected from the group consisting of Cu, Mn, Zn, Co, Ni, Mg, Ca and Fe. The method further comprises applying the formulation via at least one of a foliar application, a seed application, and a soil application.

In some embodiments of the method, Cu is present in an amount between 5 to 7% (liquid form), Mn is present in an amount between 5 to 7% (liquid form), Zn is present in an amount between 5 to 7% (liquid form), Mg is present in an amount between 1.5 to 3.5% (liquid form), Co is present in an amount between 3.5 to 5.5% (liquid form),

Ni is present in an amount between 3.5 to 5.5% (liquid form), Ca is present in an amount between 5 to 7% (liquid form), and/or Fe is present in an amount between 5 to 7% (liquid form). In some embodiments, Cu is present in an amount between 13 to 16.5% (solid form), Mn is present in an amount between 13 to 16.5% (solid form), Zn is present in an amount between 13 to 16.5% (solid form), Mg is present in an amount between 6 to 7% (solid form), Co is present in an amount between 13 to 16.5% (solid form), Ni is present in an amount between 13 to 16.5% (solid form), Ca is present in an amount between 13 to 16.5% (solid form), and/or Fe is present in an amount between 13 to 16.5% (solid form).

### BRIEF DESCRIPTION OF THE DRAWINGS

The embodiments described herein may be better understood by referring to the following description in conjunction with the accompanying drawings.

FIG. 1 is an exemplary embodiment of a reaction scheme in accordance with the present disclosure, chelate A.

FIG. 2 is an exemplary embodiment of obtained chelates in solution in accordance with the present disclosure.

FIG. 3 is an exemplary embodiment of a compatibility test in accordance with the present disclosure.

FIG. 4 is an exemplary embodiment of a corrosion test in accordance with the present disclosure.

FIG. 5 is an exemplary embodiment of Mn content of old leaves in accordance with the present disclosure.

FIG. 6 is an exemplary embodiment of Mn content of new leaves in accordance with the present disclosure.

FIG. 7 is an exemplary embodiment of Mn translocation comparing EDTA chelates and Glu+ Mal chelates in accordance with the present disclosure.

FIG. 8 is an exemplary embodiment of micronutrient guarantees of water-soluble formulations comprising a zinc chelate in accordance with the present disclosure.

FIG. 9 is an exemplary embodiment of a reaction scheme in accordance with the present disclosure, chelate B.

### DETAILED DESCRIPTION OF THE DISCLOSURE

In exemplary embodiments, the present disclosure describes a foliar nutrient product that incorporates two organic ligands forming a chelate with nutrients (e.g., copper (Cu), manganese (Mn), zinc (Zn), nickel (Ni), cobalt (Co), magnesium (Mg), calcium (Ca) and Iron (Fe)), and forming fully water-soluble metal chelates to be applied as suitable chelated nutrients in water-soluble formulations of interest. Particularly, embodiments of the present disclosure describe glutamate-malate and/or glutamate-aspartate chelates that are in general smaller than EDTA chelates and that are more concentrated with nutrients than EDTA chelates. More concentrated products are a market requirement to reduce the cost of transportation and storage. Besides that, glutamate-malate and/or glutamate-aspartate chelates have equivalent characteristics to EDTA chelates with respect to stability, pH, bonding strength, solubility, and compatibility, e.g., for use as a satisfactory EDTA chelates alternative for providing nutrients in agronomical applications.

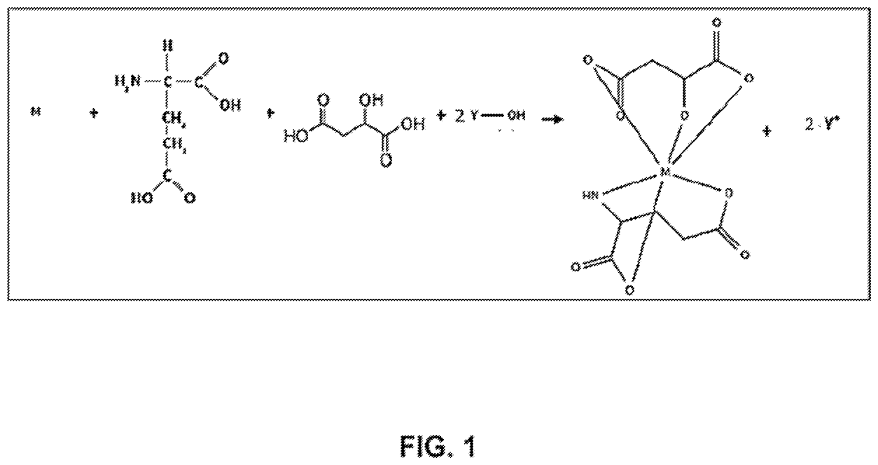

Turning now to FIG. 1, the illustrated reaction scheme depicts the general chelation reaction described herein. In exemplary embodiments, the chelates have the following structure: [M(Ligand 1)(Ligand 2)]2Y, where:
M=metal (Cu, Mn, Zn, Co, Ni, Mg, Ca and Fe) without a counter ion (for a chelation reaction, the metal is provided as a metal carbonate (CO₃²⁻) or metal oxide (O²⁻) reagent as described herein below);
Ligand 1=glutamic acid;
Ligand 2=malic acid; and
Y=base proven cation used for final solution pH adjustment (e.g., NaOH, KOH or NH4OH). 25

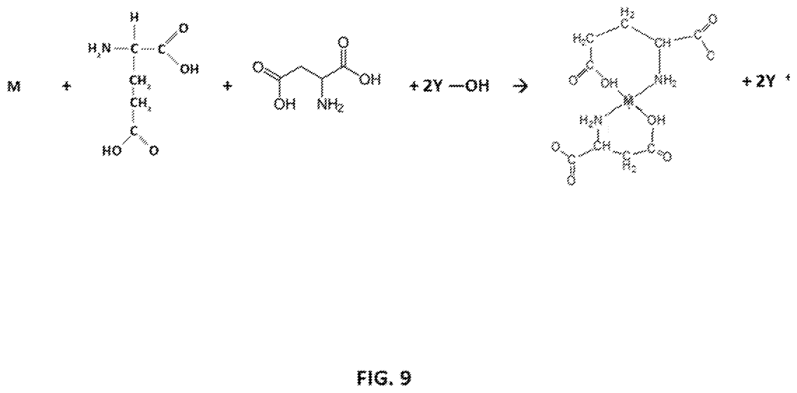

Turning now to FIG. 9, the illustrated reaction scheme depicts the general chelation reaction described herein. In exemplary embodiments, the chelates have the following structure: [M(Ligand 1)(Ligand 2)]2Y, where:
M=metal (Cu, Mn, Zn, Co, Ni, Mg, Ca and Fe) without a counter ion (for a chelation reaction, the metal is provided as a metal carbonate (CO₃²⁻) or metal oxide (O²⁻) reagent as described herein below);
Ligand 1=glutamic acid;
Ligand 2=aspartic acid; and
Y=base proven cation used for final solution pH adjustment (e.g., NaOH, KOH or NH4OH).

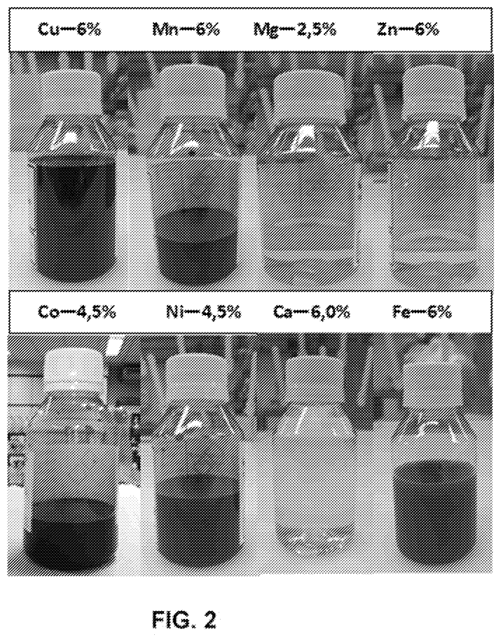

The chelation reactions were carried out with carbonate salts of Cu, Mn, Co, Ni, Mg and Ca metals and oxide for Zn and Fe metals. All salts became soluble when complexed with glutamic and malic acid as 1:1:1, metal:binder proportion (see FIG. 2), or glutamic and aspartic acid as 1:1:1, metal:binder proportion That is, a binder proportion ratio of glutamic acid to malic acid to metal is 1:1:1. FIG. 2 illustrates the obtained chelates of Cu at 6%, Mn at 6%, Mg at 2.5%, Zn at 6%, Ca at 6%, Fe at 6%, Co at 4.5%, and Ni at 4.5% in solution and/or with glutamic and aspartic acid After chelation was completed, each developed chelate (e.g., when applied as liquid or dried), was suitable for use as an isolated source of nutrient(s) or combined into water-soluble formulations that enhance the nutritional action with the insertion of other raw materials (e.g., such as the use of the Zn chelate in the formulation of Tonus® or Kellus Blindex®, registered commercial products). In exemplary embodiments, the chelates described herein are suitable for agronomical applications including foliar applications and/or drip irrigation applications.

For liquid forms, the Cu, Mn, Zn, Ca and/or Fe can be present in an amount between 5 to 7% including 5.1, 5.2, 5.3, 5.4, 5.5, 5.6, 5.7, 5.8, 5.9. 6, 6.1, 6.2, 6.3, 6.4, 6.5, 6.6, 6.7, 6.8, 6.9, and 7%. For liquid forms, the Mg can be present in an amount between 1.5 and 3.5% including 1.5, 1.6, 1.7, 1.8, 1.9, 2, 2.1, 2.2, 2.3, 2.4, 2.5, 2.6, 2.7, 2.8, 2.8, 3, 3.1, 3.2, 3.3, 3.4, and 3.5%. For liquid forms, the Co and or Ni can be present in an amount between 3.5 and 5.5% including 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, and 5.5%. Additionally, for liquid forms, the percentage(s) provided are calculated as mass solute/mass solution.

For solid forms (dried solution), the Cu, Mn, Zn, Ca, Co, Ni and/or Fe can be present in an amount between 13 to 16.5% including 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4 and16.5. For solid forms (spray dried solution), the Mg can be present in an amount between 6.0 and 7.0% including 6.1, 6.2, 6.2, 6.3, 6.4, 6.5, 6.6, 6.7, 6.8, 6.9 and 7.0%.

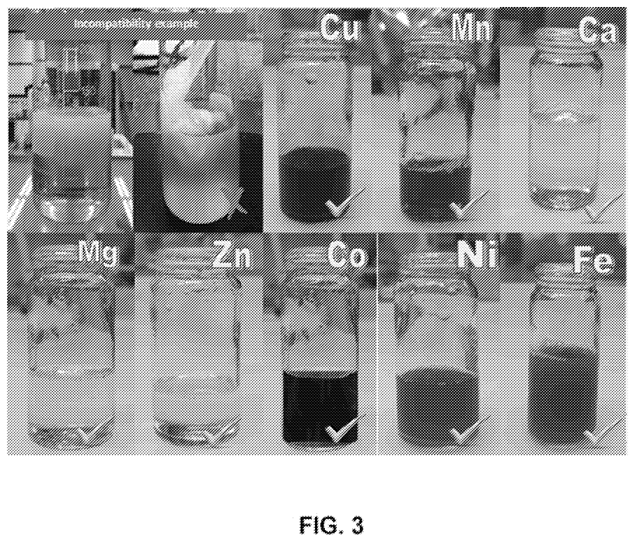

As shown in FIG. 3, compatibility tests were performed with the pesticide Agricultural Glyphosate with satisfactory physical results. Each of the Cu, Mn, Mg, Zn, Co, Ca, Fe and Ni chelates remained in solution.

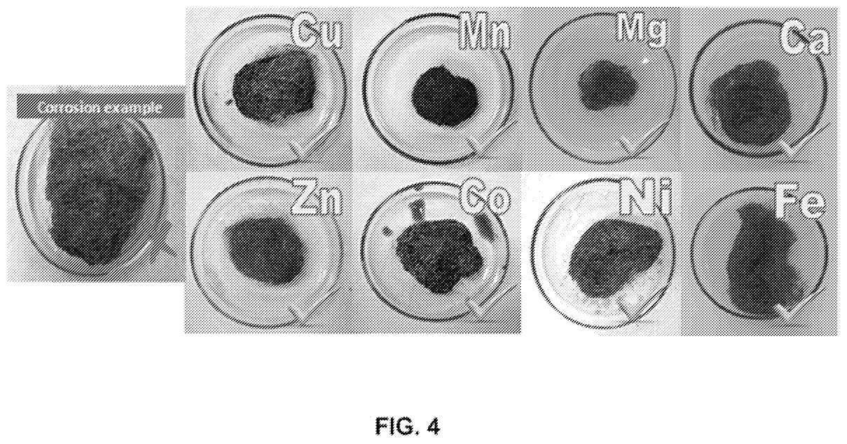

As shown in FIG. 4, corrosion tests were performed for each chelate. Assuming that a neutral product of any metal does not consume steel wool in the presence of the solution, corrosion testing on the steel wool was performed to verify that the entire structure of the chelate was completed with no free charges. (see FIG. 4).

Stability testing at different PH levels was performed and turning to Table 1, there is a large range of the chelates' stability, wherein the structure of the chelates can be considered stable.

TABLE 1
Stability pH of the chelates

| | |
|---|---|
| Chelate A | Chelate B |

| | Acid | Alkaline | Acid | Alkaline |
|---|---|---|---|---|
| Metals | medium | medium | medium | medium |
| Cu²⁺ | 0.51 | 10.86 | 0.30 | 10.7 |
| Mn²⁺ | 0.41 | 9.85 | 0.38 | 9.9 |
| Mg²⁺ | 0.41 | 9.85 | 0.39 | 9.75 |
| Zn²⁺ | 0.20 | 11.30 | 0.18 | 10.80 |
| Co²⁺ | 1.35 | 10.95 | 0.85 | 10.45 |
| Ni²⁺ | 0.20 | 10.56 | 0.18 | 10.1 |
| Ca²⁺ | 0.68 | 10.64 | 0.55 | 10.21 |
| Fe²⁺ | 1.12 | 8.59 | 1.53 | 7.95 |

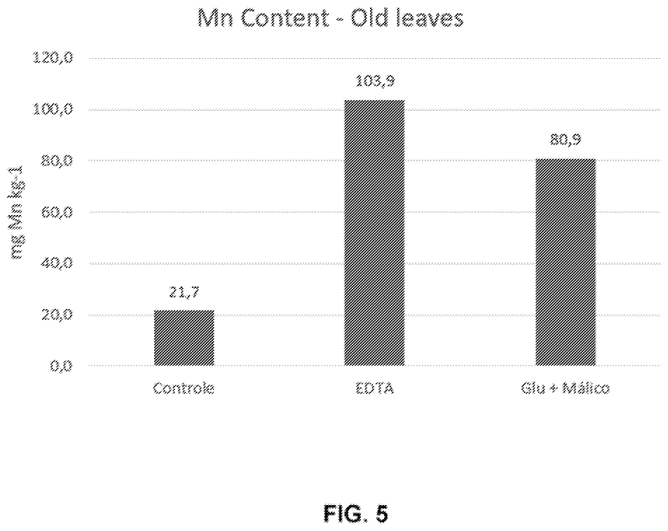

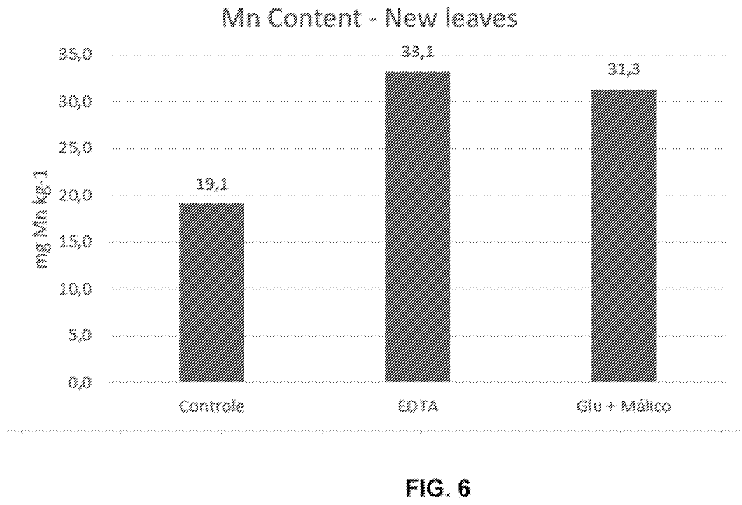

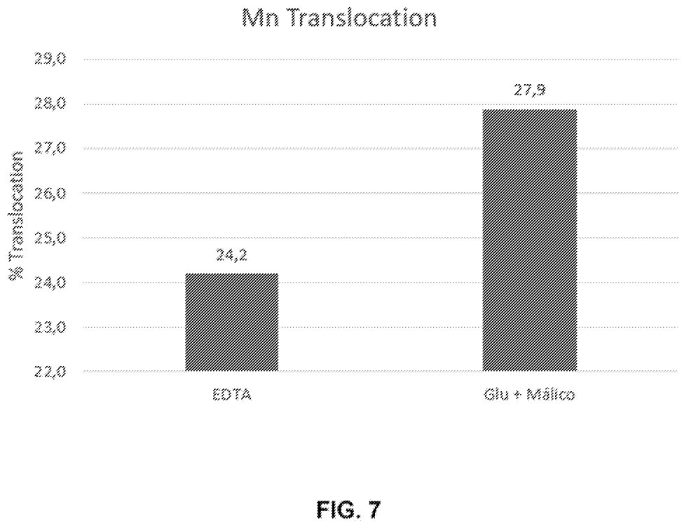

FIGS. 5 and 6 illustrate Mn content of old leaves and Mn content of new leaves, respectively. FIG. 7 shows Mn higher % of translocation which demonstrates nutrition and also metabolic recognition by dealing with organic chelating agents easily recognized by the plant.

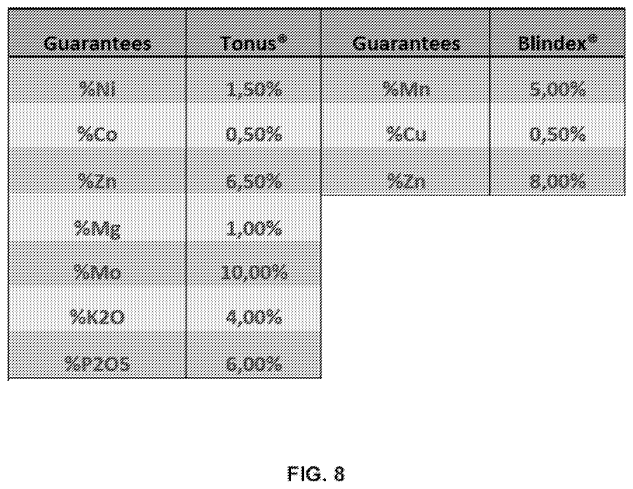

In some embodiments, the application of the chelates disclosed herein are further suitable in developing water-soluble formulations. Turning now to FIG. 8, the chart shows micronutrient guarantees of water-soluble formulations comprising a Zn chelate as described herein. For example, water-soluble formulations such as Tonus® or Blindex®, a mixture of salts including [Zn(Glu)(Mal)]2Na chelate as source of zinc and other micronutrient sources compound the guarantees exemplified in FIG. 8.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) are construed to cover both the singular and the plural, unless specifically noted otherwise. In some embodiments, the term “or” as used herein, including the claims, is used to mean “and/or” unless explicitly indicated to refer to alternatives only or to refer to the alternatives that are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and may also cover other unlisted steps. Similarly, any composition or device that “comprises,” “has” or “includes” one or more features is not limited to possessing only those one or more features and may cover other unlisted features.

All methods described herein are performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

To facilitate the understanding of the embodiments described herein, a number of terms are defined below. The terms defined herein have meanings as commonly understood by a person of ordinary skill in the areas relevant to the present disclosure. Terms such as “a,” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A chelate A having a structure [M(Ligand 1)(Ligand 2)]2Y, wherein:
M is a metal selected from the group consisting of Cu, Mn, Zn, Co, Ni, Mg, Ca and Fe;
Ligand 1 is glutamic acid;
Ligand 2 is malic acid; and
Y is a base cation, wherein the base is selected from the group consisting of NaOH KOH and/or NH4OH,
wherein a binder proportion ratio of glutamic acid to malic acid to metal is 1:1:1.

2. The chelate of claim 1, wherein at least one of the Cu, Mn, Zn, Fe and Ca are present in an amount between 5 to 7%, Mg is present in an amount between 1.5 to 3.5%, Co and Ni are present in an amount between 3.5 to 5.5% (liquid form).

3. The chelate of claim 1, wherein at least one of the Cu, Mn, Zn, Co, Ni, Fe and Ca are present in an amount between 13 to 16.5%, Mg is present in an amount between 6.0 to 7.0% (solid form).

4. A formulation for agronomical application, the formulation comprising:
a chelate A having a structure [M(Ligand 1)(Ligand 2)]2Y, wherein:
M is a metal selected from the group consisting of Cu, Mn, Zn, Co, Ni, Mg, Ca and Fe;
Ligand 1 is glutamic acid;
Ligand 2 is malic acid; and
Y is a base cation, wherein the base is selected from the group consisting of NaOH KOH and/or NH4OH; and
water,
wherein a binder proportion ratio of glutamic acid to malic acid to metal is 1:1:1.

5. The formulation of claim 4, wherein at least one of the Cu, Mn, Zn, Fe and Ca are present in an amount between 5 to 7%, Mg is present in an amount between 1.5 to 3.5%, Co and Ni are present in an amount between 3.5 to 5.5% (liquid form).

6. The formulation of claim 4, wherein at least one of the Cu, Mn, Zn, Co, Ni, Fe and Ca are present in an amount between 13 to 16.5%, Mg is present in an amount between 6.0 to 7.0% (solid form).

7. A method of applying an agronomical formulation, the method comprising:
preparing a formulation comprising a water soluble chelate A having a structure [M(Ligand 1)(Ligand 2)]2Y, wherein:
M is a metal selected from the group consisting of Cu, Mn, Zn, Co, Ni, Mg, Ca and Fe;
Ligand 1 is glutamic acid;
Ligand 2 is malic acid; and
Y is a base cation, wherein the base is selected from the group consisting of NaOH KOH and/or NH4OH;
wherein a binder proportion ratio of glutamic acid to malic acid to metal is 1:1:1; and
applying the formulation via at least one of a foliar application, a seed application, and a soil application.

8. The method of claim 7, wherein at least one of the Cu, Mn, Zn, Fe and Ca are present in an amount between 5 to 7%, Mg is present in an amount between 1.5 to 3.5%, Co and Ni are present in an amount between 3.5 to 5.5% (liquid form).

9. The method of claim 7, wherein at least one of the Cu, Mn, Zn, Co, Ni, Fe and Ca are present in an amount between 13 to 16.5%, Mg is present in an amount between 6.0 to 7.0% (solid form).

10. A chelate B having a structure [M(Ligand 1)(Ligand 2)]2Y, wherein:
M is a metal selected from the group consisting of Cu, Mn, Zn, Co, Ni, Mg, Ca and Fe;
Ligand 1 is glutamic acid;
Ligand 2 is aspartic acid; and
Y is a base cation, wherein the base is selected from the group consisting of NaOH and/or KOH; and
wherein a binder proportion ratio of glutamic acid to malic acid to metal is 1:1:1.

11. The chelate of claim 10, wherein at least one of the Cu, Mn, Zn, Fe and Ca are present in an amount between 5 to 7%, Mg is present in an amount between 1.5 to 3.5%, Co and Ni are present in an amount between 3.5 to 5.5% (liquid form).

12. The chelate of claim 10, wherein at least one of the Cu, Mn, Zn, Co, Ni, Fe and Ca are present in an amount between 13 to 16.5%, Mg is present in an amount between 6.0 to 7.0% (solid form).

13. A formulation for agronomical application, the formulation comprising:
a chelate B having a structure [M(Ligand 1)(Ligand 2)]2Y, wherein:
M is a metal selected from the group consisting of Cu, Mn, Zn, Co, Ni, Mg, Ca and Fe;
Ligand 1 is glutamic acid;
Ligand 2 is aspartic acid; and
Y is a base cation, wherein the base is selected from the group consisting of NaOH KOH and/or NH4OH; and
water;
wherein a binder proportion ratio of glutamic acid to malic acid to metal is 1:1:1.

14. The formulation of claim 13, wherein at least one of the Cu, Mn, Zn, Fe and Ca are present in an amount between 5 to 7%, Mg is present in an amount between 1.5 to 3.5%, Co and Ni are present in an amount between 3.5 to 5.5% (liquid form).

15. The formulation of claim 13, wherein at least one of the Cu, Mn, Zn, Co, Ni, Fe and Ca are present in an amount between 13 to 16.5%, Mg is present in an amount between 6.0 to 7.0% (solid form).

16. A method of applying an agronomical formulation, the method comprising:
preparing a formulation comprising a water soluble chelate B having a structure [M(Ligand 1)(Ligand 2)]2Y, wherein:
M is a metal selected from the group consisting of Cu, Mn, Zn, Co, Ni, Mg, Ca and Fe;
Ligand 1 is glutamic acid;
Ligand 2 is aspartic acid; and
Y is a base cation, wherein the base is selected from the group consisting of NaOH KOH and/or NH4OH;
wherein a binder proportion ratio of glutamic acid to malic acid to metal is 1:1:1; and
applying the formulation via at least one of a foliar application, a seed application, and a soil application.

17. The method of claim 16, wherein at least one of the Cu, Mn, Zn, Fe and Ca are present in an amount between 5 to 7%, Mg is present in an amount between 1.5 to 3.5%, Co and Ni are present in an amount between 3.5 to 5.5% (liquid form).

18. The method of claim 16, wherein at least one of the Cu, Mn, Zn, Co, Ni, Fe and Ca are present in an amount between 13 to 16.5%, Mg is present in an amount between 6.0 to 7.0% (solid form).

19. A formulation for agronomical application, the formulation comprising a mixture of a chelate A as defined in claim 1 and a chelate B as defined in claim 10.

20. The formulation of claim 19, wherein at least one of the Cu, Mn, Zn, Fe and Ca are present in an amount between 5 to 7%, Mg is present in an amount between 1.5 to 3.5%, Co and Ni are present in an amount between 3.5 to 5.5% (liquid form).

21. The formulation of claim 19, wherein at least one of the Cu, Mn, Zn, Co, Ni, Fe and Ca are present in an amount between 13 to 16.5%, Mg is present in an amount between 6.0 to 7.0% (solid form).

22. A method of preparing a formulation for agronomical application comprising mixing a chelate A as defined in claim 1 and a chelate B as defined in claim 10.

23. The method of claim 22, wherein at least one of the Cu, Mn, Zn, Fe and Ca are present in an amount between 5 to 7%, Mg is present in an amount between 1.5 to 3.5%, Co and Ni are present in an amount between 3.5 to 5.5% (liquid form).

24. The method of claim 22, wherein at least one of the Cu, Mn, Zn, Co, Ni, Fe and Ca are present in an amount between 13 to 16.5%, Mg is present in an amount between 6.0 to 7.0% (solid form).
